(19) United States
(12) Patent Application Publication
(10) Pub. No.: US 2023/0157631 A1
(43) Pub. Date: May 25, 2023

(54) SYSTEMS AND METHODS FOR MONITORING AND CONTROL OF SLEEP PATTERNS

(71) Applicant: Monash University, Clayton, Victoria (AU)

(72) Inventors: Tracey Sletten, Victoria (AU); Prerna Varma, Victoria (AU); Andrew James Tucker, Victoria (AU); Andrew John Kelvin Phillips, Victoria (AU); Benjamin Irwin Shelly, Pittsburgh, PA (US); Gary Nelson Garcia Molina, Madison, WI (US); Ilankaikone Senthooran, Victoria (AU); Jade Mary Murray, Victoria (AU); Jesse Salazar, Gibsonia, PA (US); Lauren Adele Booker, Victoria (AU); Mark Erskine Howard, Victoria (AU); Michelle MaGee, Victoria (AU); Monica Helen Bush, Murrysville, PA (US); Shanthakumar Madhan Wilson Rajaratnam, Victoria (AU); Svetlana Postnova, New South Wales (AU); Yash Mokashi, Pittsburgh, PA (US)

(21) Appl. No.: 17/799,206

(22) PCT Filed: Feb. 12, 2021
(86) PCT No.: PCT/AU2021/050126

§ 371 Date: Aug. 11, 2022

Related U.S. Application Data
(60) Provisional application No. 62/976,278, filed on Feb. 13, 2020.

(51) Int. Cl. A61B 5/00; G16H 50/20; G16H 10/20

(52) U.S. Cl. CPC A61B 5/4815; G16H 50/20; G16H 10/20

(57) ABSTRACT

Described embodiments generally relate to a method for improving data accuracy of sleep pattern data. The method comprises receiving first data relating to at least one sleep pattern metric; receiving second data relating to the at least one sleep pattern metric, wherein the second data is data entered by a user; determining the difference between the first data and the second data to calculate a data infidelity value; and in response to the data infidelity value exceeding a predetermined threshold, prompting a user to enter third data relating to at least one metric.

### TECHNICAL FIELD

Embodiments generally relate to systems and methods for monitoring and control of sleep patterns. Specifically, embodiments relate to systems and methods for assisting in reducing the effect of sleep disorders.

### BACKGROUND

Many employees are permanently assigned to working overnight or alternating shifts, particularly in industries such a nursing, mining, and transport. For example, around 20 million US workers were assigned to shift work in 2019. When interviewed, 75% of these shift workers felt like they have little control over their sleep routines, and were worried about the health consequences of lack of sleep. Shift work has been shown to be a risk factor for health problems by disrupting circadian rhythms, which may increase the probability of developing cardiovascular disease, cognitive impairment, diabetes, and obesity, among other conditions. Furthermore, shift work often contributes to strain in marital, family and personal relationships. While some techniques exist for assisting shift workers to manage their sleep patterns and avoid sleep disorder conditions, workers may struggle to find techniques that are effective in their particular scenario, and may find it difficult to comply with the techniques over long periods of time.

It is desired to address or ameliorate one or more shortcomings or disadvantages associated with prior systems and methods for monitoring and controlling sleep patterns, or to at least provide a useful alternative thereto.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this document, a statement that an element may be “at least one of” a list of options is to be understood to mean that the element may be any one of the listed options, or may be any combination of two or more of the listed options.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

Some embodiments relate to a method for improving data accuracy of sleep pattern data, the method comprising:
receiving first data relating to at least one sleep pattern metric;
receiving second data relating to the at least one sleep pattern metric, wherein the second data is data entered by a user;
determining the difference between the first data and the second data to calculate a data infidelity value; and
in response to the data infidelity value exceeding a predetermined threshold, prompting a user to enter third data relating to at least one metric.

According to some embodiments, the first data is data entered by a user. In some embodiments, the first data is sensor data received from at least one sensor.

Some embodiments further comprise determining the difference between the first data, the second data and the third data to calculate an updated data infidelity value; and in response to the updated data infidelity value exceeding a predetermined threshold, repeating the steps of prompting the user to enter further data or confirming already submitted data and calculating the updated data infidelity value until the updated data infidelity value does not exceed the predetermined threshold.

Some embodiments further comprise prompting a user to enter second data relating to at least one metric, wherein the second data is received in response to the prompt.

According to some embodiments, the prompting comprises presenting the user with a question, and the second data is based on the user's response to the question.

In some embodiments, the second data is data received from a remote device comprising at least one sensor.

In some embodiments, prompting the user to enter third data comprises presenting a modified question to the user, the modified question being based on a question previously presented to the user and having the same semantic meaning as the question previously presented to the user.

Some embodiments further comprise generating the modified question based on the question previously presented to the user using natural language processing techniques.

Some embodiments further comprise retrieving the modified question from a database of questions.

Some embodiments further comprise processing the first data and the second data to map the data to the at least one sleep pattern metric.

According to some embodiments, the at least one sleep pattern metric comprises at least one of a time in bed metric, a total sleep time metric, a wake after sleep onset (WASO) metric, a sleep onset latency (SOL) metric, and a sleep efficiency metric.

Some embodiments further comprise using at least one of the first data, second data and third data to determine a value for the at least one sleep pattern metric.

Some embodiments further comprise generating a sleep pattern recommendation for presenting to the user based on the determined value of the sleep pattern metric.

Some embodiments further comprise prompting the user to confirm the accuracy of at least one of the first data, second data and third data.

Some embodiments further comprise tracking any questions presented to the user that result in the user providing data having a high data infidelity value, to determine questions that lack clarity.

Some embodiments further comprise rewording any questions that result in the user providing data having a high data infidelity value.

Some embodiments further comprise tracking word combinations within questions presented to the user that result in the user providing data having a high data infidelity value, to determine word combinations that lack clarity.

Some embodiments relate to a method for presenting sleep pattern recommendations to a user, the method comprising:
receiving sleep pattern data from a population;
performing clustering of the received sleep pattern data;
receiving sleep pattern data from a user;
identifying a cluster that is most closely associated with the sleep pattern data received from the user;
receiving a plurality of sleep pattern recommendations to provide to the user; retrieving a sleep pattern recommendation order based on the identified cluster; and
ordering the plurality of sleep pattern recommendations based on the retrieved sleep pattern recommendation order.

Some embodiments further comprise presenting at least one of the plurality of sleep pattern recommendations to the user according to the retrieved sleep pattern recommendation order.

In some embodiments, the plurality of sleep pattern recommendations are presented to the user simultaneously.

According to some embodiments, the plurality of sleep pattern recommendations are presented to the user sequentially.

Some embodiments further comprise presenting the at least one of the plurality of sleep pattern recommendations to the user alongside a degree of effectiveness of the recommendation.

According to some embodiments, prompting the user to enter data relating to an effectiveness of the at least one recommendation may comprise prompting the user to enter data relating to at least one of the user's waking mood, alertness and sleepiness after having adopted the at least one recommendation.

Some embodiments further comprise pre-processing the sleep pattern data received from the user into a normalised data vector.

In some embodiments, the clustering is performed using an agglomerative clustering technique.

In some embodiments, the clustering is performed using at least one of partitioning clustering, k-means clustering and hierarchical clustering.

Some embodiments further comprise masking the recommendations based on user data to avoid presenting the user with irrelevant or infeasible recommendations.

Some embodiments further comprise providing the user with an alternative recommendation to replace at least one masked recommendation.

Some embodiments further comprise prompting the user to enter data relating to an effectiveness of the at least one recommendation.

Some embodiments further comprise using the entered data to modify the sleep pattern recommendation order associated with the identified cluster.

In some embodiments, the sleep pattern recommendations are generated according to the method of some other embodiments.

Some embodiments relate to a method for improving sleep patterns in users, the method comprising:
receiving data relating to at least one sleep pattern metric from a first remote device;
processing the data to generate at least one sleep pattern recommendation;
processing the data to generate at least one instruction to a second remote device, to cause the second remote device to implement the recommendation;
displaying the at least one recommendation to the user; and
sending the at least one instruction to the second remote device.

Some embodiments further comprise pre-processing the data received from the first remote device to format the data to a common data format.

Some embodiments further comprise deriving at least one sleep pattern parameter from the data.

In some embodiments, processing the data to generate at least one sleep pattern recommendation comprises using a decision tree.

According to some embodiments, processing the data to generate at least one sleep pattern recommendation comprises using a model driven recommendation model.

According to some embodiments, the model driven recommendation model uses at least one of a bio-mathematical model and a biophysical model.

In some embodiments, the model uses a system of ordinary differential equations.

In some embodiments, the differential equations are based on neurobiological mechanisms of sleep and circadian regulation.

In some embodiments, the first remote device comprises at least one of a home monitoring hub, a car monitoring hub, a recovery system, a wearable device, a smart cup, an augmented reality device, a virtual reality device, a biological data device, a bed partner input device, an emotion detection system, a manual entry system, a light sensor and a work place monitoring hub.

According to some embodiments, the second remote device comprises at least one of a change coaching system, a calendar input system, an augmented reality device, a virtual reality device, an engagement system, a biological feedback system, a home automation system, a communication system, a behaviour recommendation system, a long term connection system, and a car.

In some embodiments, processing the data to generate at least one sleep pattern recommendation is performed according to the method of some other embodiments.

In some embodiments, displaying the at least one recommendation to the user is performed according to the method of some other embodiments.

Some embodiments relate to a machine-readable medium storing non-transitory instructions which, when executed by one or more processors, cause an electronic apparatus to perform the method of some other embodiments.

Some embodiments relate to an apparatus, comprising processing circuitry and a machine-readable medium storing non-transitory instructions which, when executed by the processing circuitry, cause the apparatus to perform the method of some other embodiments.

### BRIEF DESCRIPTION OF DRAWINGS

Embodiments are described in further detail below, by way of example and with reference to the accompanying drawings, in which:

FIG. 1 illustrates a sleep pattern system according to some embodiments;

FIG. 2 shows a flowchart illustrating a method of sleep pattern management performed by the system of FIG. 1;

FIG. 3 shows a flowchart illustrating a method of improving data accuracy for sleep pattern management performed by the system of FIG. 1;

FIG. 4 shows a table illustrating the mapping of questions to metrics as performed by the system of FIG. 1;

FIG. 5 shows a table illustrating fidelity and redundancy processing as performed by the system of FIG. 1;

FIG. 6 shows a table illustrating infidelity mapping as performed by the system of FIG. 1;

FIG. 7 shows a table illustrating high infidelity word-combination identification as performed by the system of FIG. 1;

FIG. 8 shows a flowchart illustrating a method of improving the presentation of recommendations for sleep pattern management performed by the system of FIG. 1;

FIG. 9 shows a table illustrating an example of recommendations displayed by the system of FIG. 1;

FIG. 10 shows a diagram illustrating sleep pattern management functions performed by the system of FIG. 1;

FIG. 11 shows a data collection process performed by the system of FIG. 1; and

FIG. 12 shows a timeline illustrating a potential set of outputs delivered by the system of FIG. 1.

### DETAILED DESCRIPTION

Embodiments generally relate to systems and methods for monitoring and control of sleep patterns. Specifically, embodiments relate to systems and methods for assisting in reducing the effect of sleep disorders.

Shift workers are at increased risk of a number of detrimental functional and health outcomes. Specifically, shift workers commonly deal with unconventional work hours, which can lead to shift work sleep disorders such as chronic sleep disturbance, as well as other health conditions such as gastrointestinal problems, cardiovascular disease, mood and affect disorders and cancer. These may arise as a result of misalignment between the endogenous circadian pacemaker of the shift worker and their sleep-wake patterns. Shift work sleep disorders particularly affect workers on rotating shifts, shifting between day and night shifts during a work week, and users with complex living situations such as users with families, children and partners. Development of personalized sleep-wake management systems is critical to improving sleep-wake and functional outcomes for shift workers. However, while a number of recommendations and suggestions for handling shift work exist, it can be hard for shift workers to find the information that is relevant to them and that would be most useful in helping them cope with their individual schedule and circumstances.

While some sleep scheduling systems exist, existing systems require the user to input shifts manually every time, and do not automatically upload, update, and share shift schedules, or provide coaching support for sleep, mood, or alertness. Sleep, mood and alertness are three areas that are commonly affected by shift work schedules. Furthermore, sleep pattern management systems tend to require tactile interaction with a mobile phone, tablet, or computer. They do not integrate with other devices like wearables or smart home devices. As a result, inputting information about shift schedules and life commitments manually every time can be arduous, and the input information can be inaccurate.

It would be helpful to provide shift workers with a sleep pattern management tool to offer them help in the form of instructions or recommendations that would assist them in dealing with their unconventional working hours and improve their recovery time for working shifts, to avoid shift work sleep disorders and to assist shift workers with raising their mood and alertness levels for family and work requirements. In particular, it would be helpful to provide shift workers with personalized recommendations.

According to some embodiments, this personalization is done through algorithms that take into account user's responses to an on-boarding questionnaire or survey. Some embodiments relate to systems and methods for reducing the answer infidelity of user responses to such a questionnaire or survey, to improve the accuracy of the algorithm outcome and in turn the effectiveness of the recommendations.

Some embodiments relate to systems and methods for ranking a set of recommendations provided to a user based on a profile of that user. In particular, some embodiments relate to systems and methods for ranking a set of recommendations for providing to a user relating to shift work and sleep patterns.

Some embodiments relate to systems and methods for providing users with assistance during shift work based on received data, including subjective and objective data.

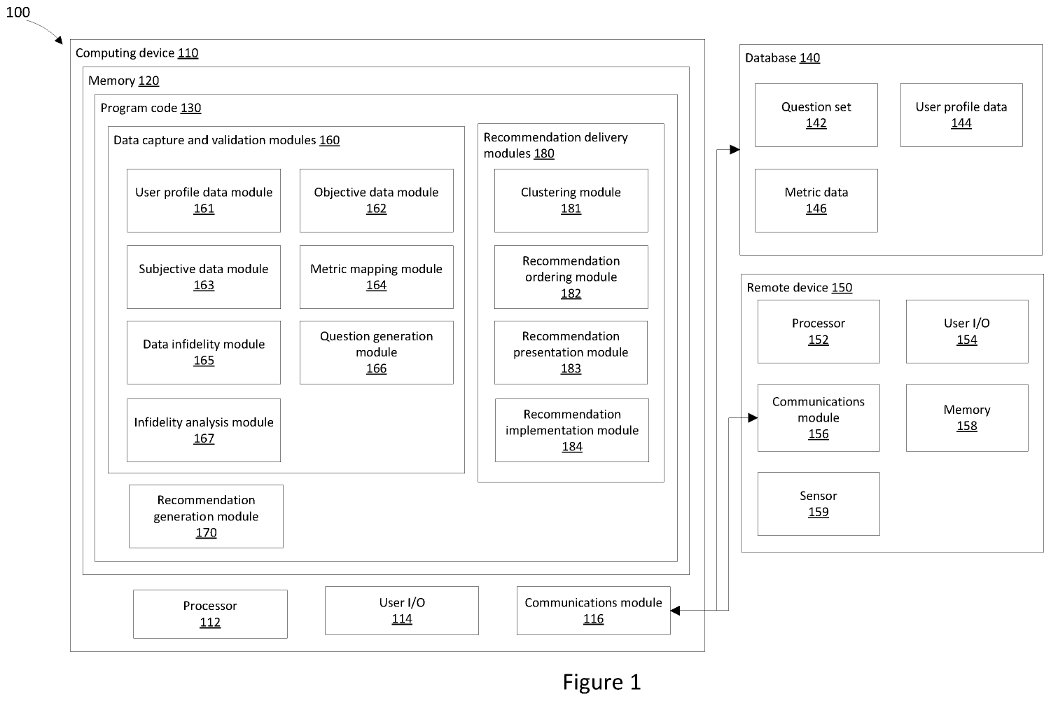

FIG. 1 illustrated a sleep pattern management system 100. Sleep pattern management system 100 comprises a computing device 110 having a processor 112, and memory 120. Computing device 110 may comprise one or more computers, servers, or other computing devices, and may be a distributed server system or a cloud based computing system in some embodiments. According to some embodiments, computing device 110 may be a smart phone, wearable, laptop, or desktop computer. Processor 112 may comprise one or more microprocessors, central processing units (CPUs), application specific instruction set processors (ASIPs), or other processors capable of reading and executing instruction code.

Memory 120 may comprise one or more volatile or non-volatile memory types, such as RAM, ROM, EEPROM, or flash, for example. Memory 120 may store data accessible to processor 112. Memory 120 may further store program code 130 executable by processor 112 to perform sleep pattern management functions. Program code 130 may comprise a number of code modules relating to sleep pattern management, which together may form a sleep pattern management application or program. For example, in the illustrated embodiment, program code 130 comprises a number of code modules relating to sleep pattern management functions, including data capture and validation modules 160, recommendation generation module 170, and recommendation delivery modules 180.

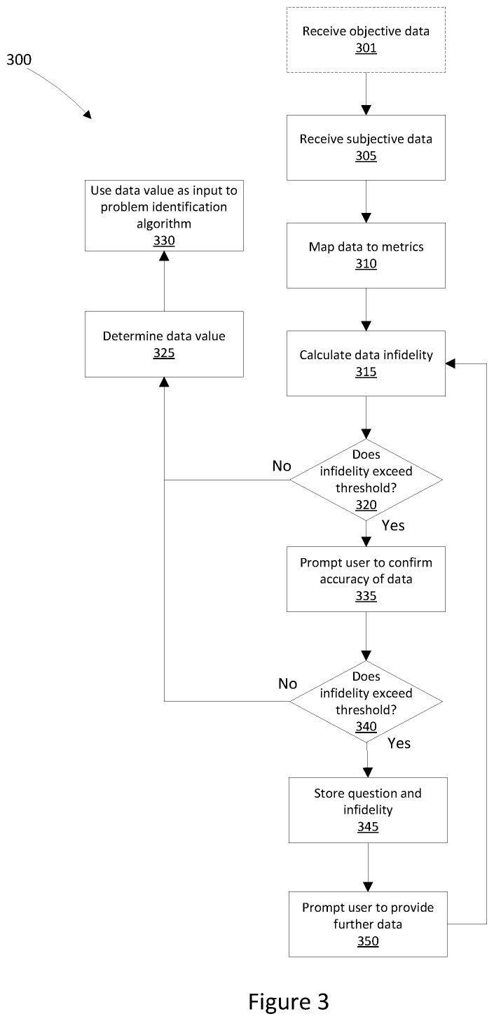

Data capture and validation modules 160, when executed by processor 112, may be configured to cause processor 112 to perform a number of data capture and validation functions. For example, executing user profile data module 161 may cause processor 112 to perform functions relating to the capture and processing of user data, such as the name, age, and gender of a user of device 110, for example. Executing objective data module 162 may cause processor 112 to capture and process objective data collected by device 110 or received from a remote computing device, such as the distance travelled by device 110, or the duration for which a screen of device 110 was turned on over a period of time, for example. Executing subjective data module 163 may cause processor 112 to capture and process subjective data received from the user of device 110, such as how the user feels, or how many times the user recalls waking up overnight, for example. Executing metric mapping module 164 may cause processor 112 to map objective and subjective data received during execution of modules 162 and 163 to one or more predetermined metrics. Executing data infidelity module 165 may cause processor 112 to determine a fidelity or infidelity of objective and subjective data received during execution of modules 162 and 163. Executing question generation module 166 may cause processor 112 to generate questions or prompts to present to the user, to prompt the user to enter responses to be captured as subjective data by executing subjective data module 163. Executing infidelity analysis module 167 may cause processor 112 to perform analysis functions relating to the infidelity of received data, to identify causes for the data infidelity. The functions of modules 161 to 167 are described in further detail below with reference to FIGS. 3 to 7.

Recommendation generation module 170, when executed by processor 112, may cause processor 112 to generate sleep pattern recommendations to a user, based on subjective and objective data received and validated by modules 160. The recommendations may include recommended sleep and wake times, caffeine intake recommendations, and light exposure and avoidance recommendations, for example.

Recommendation delivery modules 180, when executed by processor 112, may be configured to cause processor 112 to perform a number of functions relating to the delivery of recommendations generated by module 170. For example, executing clustering module 181 may cause processor 112 to perform clustering of user data, to identify a user profile type most closely associated with the user of device 110. Executing recommendation ordering module 182 may cause processor 112 to arrange the recommendations generated by module 170 by order of likely effectiveness, based on the user profile type determined by module 181. Executing recommendation presentation module 183 may cause processor 112 to present the recommendations generated by module 170 in the order determined by module 182. Executing recommendation implementation module 184 may cause processor 112 to implement recommendations generated by module 170, where those recommendations can be implemented by device 110 or by a remote device with which device 110 can communicate.

Computing device 110 further comprises user input/output 114. User input/output 114 may comprise one or more forms of user input and/or output devices, such as one or more of a screen, keyboard, mouse, touch screen, microphone, speaker, camera, or other device that allows information to be delivered to or received from a user. User I/O 114 may be used to deliver questions and prompts to a user, such as questions and prompts generated by module 166. User I/O 114 may also be used to receive responses and information from a user, such as responses and information delivered to modules 161 and 163. User I/O 114 may also be used to deliver recommendations and information to a user, such as recommendations generated by module 170, and information based on the recorded data such as their sleep and wake times.

Computing device 110 also includes communications module 116. Communications module 116 may be configured to communicate with one or more external or remote computing devices or computing systems, via a wired or wireless communication protocol. For example, communications module 116 may facilitate communication via at least one of Wi-Fi, Bluetooth, Ethernet, USB, or via a cellular network in some embodiments.

In the illustrated embodiment, computing device 110 is in communication with database 140 and at least one remote device 150 via communication module 116. Database 140 may be a remote database storing data including a question set 142, user profile data 144 and metric data 146. Question set 142 may comprise a series of questions for presenting to a user to prompt the user to supply user profile data and subjective data, and may include questions regarding their name, age, gender, line of work, shift work schedules, sleep patterns (including napping behaviour), mood, alertness, caffeine intake, light exposure, exercise time and other lifestyle and wellbeing topics. According to some embodiments, processor 112 executing questions generation module 166 may cause communications module 116 to retrieve questions from question set 142 to present to the user of device 110. User profile data 144 may store profile data for a number of users, including the user of device 110, and may include information such as user names, ages and genders, for example. Metric data 146 may store metrics relating to sleep patterns, shift work, lifestyle and wellbeing. Metrics stored in metric data 146 may include duration of sleep, sleep latency, quality of sleep, sleep state prior to waking-up, sleep stage durations, mood and caffeine intake, for example.

Remote device 150 may comprise one or more computers, servers, or other computing devices, and may be a distributed server system or a cloud based computing system in some embodiments. According to some embodiments, remote device 150 may be a smart phone, wearable, laptop, home assistance device or desktop computer. Remote device 150 comprises a processor 152, and memory 158. Processor 152 may comprise one or more microprocessors, central processing units (CPUs), application specific instruction set processors (ASIPs), or other processor capable of reading and executing instruction code. Memory 158 may comprise one or more volatile or non-volatile memory types, such as RAM, ROM, EEPROM, or flash, for example. Memory 158 may store data and program code accessible to processor 152.

Remote device 150 further comprises user input/output 154. User input/output 154 may comprise one or more forms of user input and/or output devices, such as one or more of a screen, keyboard, mouse, touch screen, microphone, speaker, camera, or other device that allows information to be delivered to or received from a user. Remote device 150 also includes communications module 156. Communications module 156 is configured to communicate with computing device 110 via a wired or wireless communication protocol. For example, communications module 156 may facilitate communication via at least one of Wi-Fi, Bluetooth, Ethernet, USB, or via a cellular network in some embodiments.

Remote device 150 may optionally further comprise at least one sensor 159. Sensor 159 may comprise one or more of a microphone, camera, light sensor, thermometer, or accelerometer, in some embodiments. Processor 152 executing program code stored in memory 158 may be configured to receive data generated by sensor 159, and to communicate the data to computing device 110, to be processed by processor 112 executing objective data module 162.

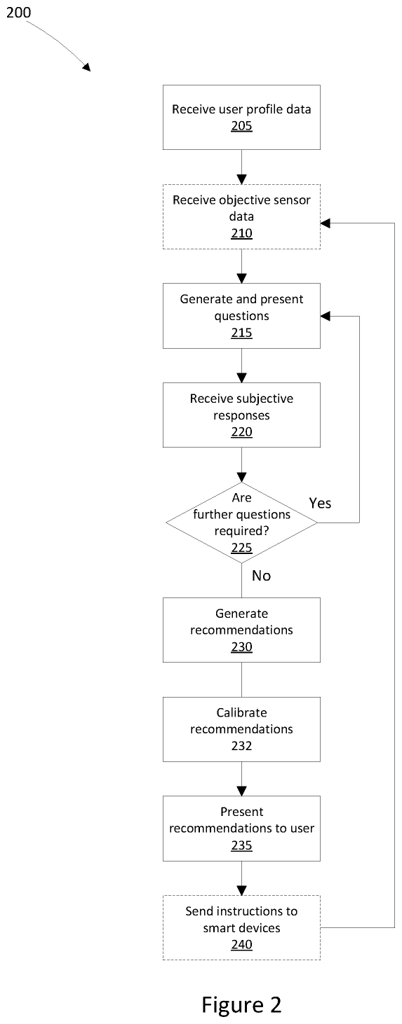

According to some embodiments, system 100 may be configured to provide sleep pattern management functions to a user who is a shift worker. The functions may relate to helping the user manage their shift work, their sleep patterns, their mood, their alertness, and generally to manage their lifestyle and wellbeing, as described below with reference to FIG. 2.

FIG. 2 illustrated a method 200 of shift management performed by system 100. At step 205, user profile data is received by processor 112 of device 110 executing user profile data module 161. Processor 112 executing module 161 may be caused to prompt the user to enter user data via user I/O 114. The data may include the user's name, age, gender, shift schedules, line of work and work cycles, for example. The data may further include information about the time zone(s) in which the user lives and/or works. The data received at step 205 may include subjective and objective data provided by the user according to some embodiments. Processor 112 may store the received data locally within memory 120, and/or may communicate the data via communications module 116 to database 140, to be stored within user profile data 144.

At optional step 210, device 110 receives objective sensor data which is stored in memory 120 and processed by processor 112 executing objective data module 162. Objective data may include data generated by sensor 159 and received from remote device 150 via communications module 116, or objective data generated by device 110, such as data relating to a frequency of use of device 110, for example. Data received from remote device 150 may include objective data such as sleep monitoring data generated by a wearable device, device usage data generated by a smart device such as a television, or other objective sensor data. According to some embodiments, the objective data received may include ongoing data captured periodically or continuously. While step 210 is illustrated as being after step 205, these steps may be performed simultaneously, or step 210 may be performed before step 205 in some embodiments.

At step 215, processor 112 of device 110 is configured to execute subjective data module 163 to prompt the user for subjective data. The subjective data may include data regarding their sleep habits, mood, and other lifestyle data. The prompts presented to the user may include questions such as what time the user went to sleep last night, how much caffeine the user consumed, or how tired the user feels, for example. The questions may be retrieved from question set 142 of database 140.

According to some embodiments, the questions retrieved from database 140 may include general questions relating to sleep habits, caffeine intake, and napping habits, for example. According to some embodiments, questions may also be retrieved based on a user schedule that the user may store on device 110 in the form of a calendar or diary. For example, retrieved questions may relate to a user's commute time, or the time it takes the user to get ready in the morning. According to some embodiments, the questions presented may be selected based on other aspects of data received, such as user profile data or objective sensor data.

According to some embodiments, processor 112 may be configured to perform step 215 periodically to prompt the user for subjective data automatically at certain times of day, which may be based on a user schedule, or historical data regarding the usage of device 110. For example, the first usage of the day of device 110 by the user may cause processor 112 to prompt the user to answer retrieved questions about what time the user went to bed and woke up. According to some embodiments, processor 112 may be configured to prompt the user to answer questions regarding caffeine intake half an hour before the user's scheduled bedtime. According to some embodiments, processor 112 may be configured to prompt a user to answer questions regarding how tired or how alert the user feels periodically throughout the day, which may be every 2 hours during the user's scheduled waking hours, for example. While step 215 is illustrated as being after steps 205 and 210, these steps may be performed simultaneously, or step 215 may be performed before either or both of steps 205 and 210 in some embodiments.

At step 220, subjective responses to the presented questions are received by processor 112 executing subjective data module 163 and stored in memory 120. According to some embodiments, the responses may be received by a user entering data using user I/O 114. The data may include their name, age, gender, shift schedules and work cycles, for example.

At step 225, processor 112 may determine whether further questions should be presented to the user. This may be determined by processor 112 executing code modules 164, 165, and 166, as described in further detail below with reference to FIGS. 3 to 7. If processor 112 determines that further questions are required, processor 112 then repeats executing step 215 to generate and present the further questions. If not, processor 112 moves to executing step 230.

At step 230, processor 112 executes recommendation generation module 170 to generate recommendations to present to a user, and store the recommendations to memory 120. The recommendations may be generated based on one or more of the user profile data, objective data and subjective data as received by processor 112 when executing modules 161, 162 and 163. According to some embodiments, at step 230 processor 112 may also generate feedback to present to the user. The feedback may include feedback on how well the user is complying with the recommendations, or how the user's mood or alertness have changed since implementing the recommendations. According to some embodiments, the feedback may include a recovery score.

At step 232, processor 112 is configured to calibrate the recommendations generated at step 230. Calibration may include identifying where automated recommendations are irrelevant or infeasible and need an adjustment. For example, if a recommendation of “consume less caffeine after 7 pm” is generated but the user has indicated they don't drink any caffeine, this recommendation may be removed. This may be considered a masking step, as described in further detail below with reference to step 830 of method 800. Processor 112 may execute some basic logic steps to adapt the recommendations to the user's circumstance before they are presented to the user, and may be configured to present updated or alternative recommendations in an attempt to increase compliance with the recommendations and/or to improve one or more aspects of the user's sleep, mood or health.

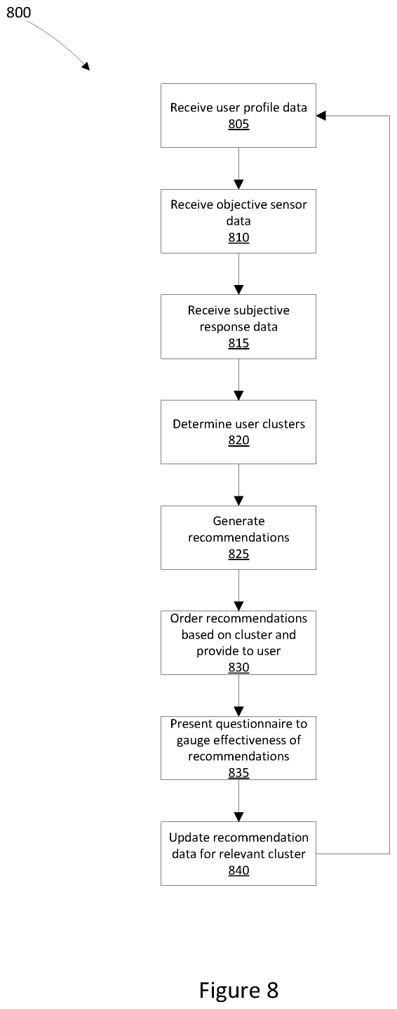

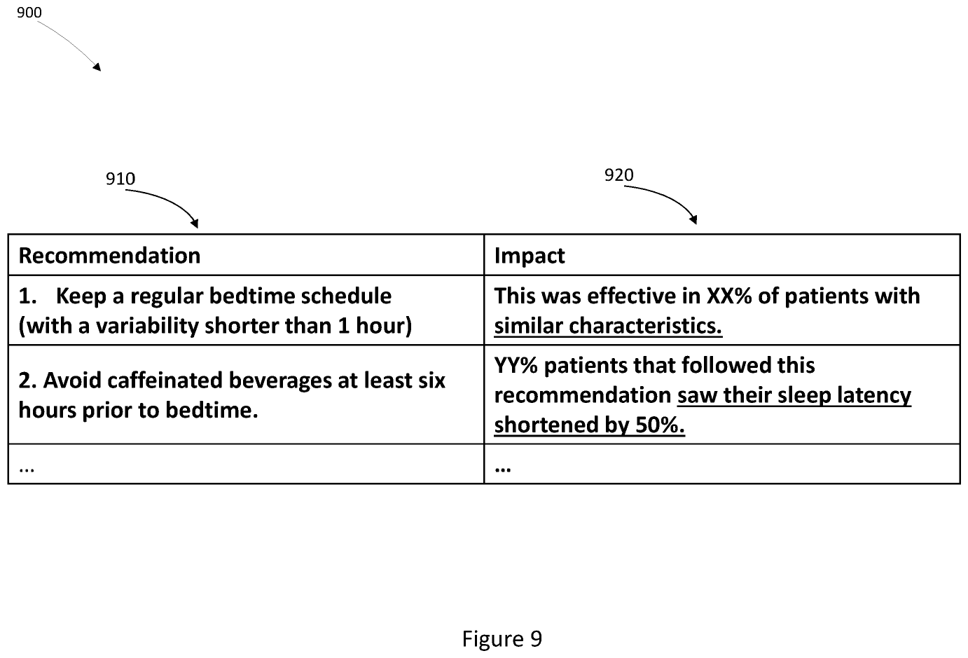

At step 235, processor 112 executes recommendation delivery modules 180 to present recommendations to the user. According to some embodiments, more than one recommendation may be generated by processor 112 executing recommendation generation module 170, and processor 112 may execute recommendation ordering module 182 to determine an order to present the recommendations to the user, as described in further detail below with reference to FIGS. 8 and 9. According to some embodiments, processor 112 may also present other data to the user, such as the feedback generated at step 230. For example, processor 112 may be configured to present data relating to the user's sleep-wake behaviour or mood data, which may be presented in the form of a recovery score. According to some embodiments, this information may be displayed visually, such as in a graphical format.

At optional step 240, processor 112 executing recommendation presentation module 183 may communicate instructions to remote smart devices, such as device 150, based on the generated recommendations produced by processor 112 at step 230. For example, where remote device 150 is a smart television, processor 112 may send instructions to remote device 150 to cause remote device 150 to dim its screen after a particular time of day, to assist the user with decreasing the amount of screen light they are subjected to during periods of the day prior to sleep.

Having completed step 235 and optionally step 240, processor 112 continues to execute method 200 from step 210, awaiting further objective sensor data and periodically prompting the user to respond to questions with objective responses. According to some embodiments, the questions presented to the user in subsequent iterations of step 215 may include questions aimed at determining the extent to which the user is complying with the recommendations presented at step 235. According to some embodiments, the questions presented to the user in subsequent iterations of step 215 may include questions aimed at determining the extent to which the recommendations presented at step 235 are having a positive effect on one or more aspects of the user's sleep, mood or health. These types of questions are discussed in further detail below with respect to FIG. 8.

According to some embodiments, in subsequent iterations of steps 230 and 232, processor 112 may be configured to present updated or alternative recommendations to the user compared to the recommendations that had previously been presented and that the user has tried to implement. This may be done where previous recommendations have been determined to be less effective, or where a user has struggled to comply with the recommendations, in an attempt to increase compliance with the recommendations and/or to improve one or more aspects of the user's sleep, mood or health, as described in further detail below with reference to step 840 of method 800.

According to some embodiments, where compliance with recommendations is determined to be low or where it is otherwise desirable to increase such compliance, processor 112 may facilitate the provision of incentives to users to further encourage them to comply with the recommendations. Incentives may be digital, such as discount codes or access to digital media, or may be physical. In the case of physical incentives, processor 112 may be configured to facilitate in communicating the relevant compliance data to a third party who may be responsible for providing the incentives.

According to some embodiments, system 100 may be used by shift workers in a number of varying industries, and may be modified to suit the needs of each particular industry.

According to some embodiments, system 100 may be configured to be used by healthcare professionals such as nurses. In this case, system 100 may be particularly configured to adapt to the highly variable shift schedules of healthcare workers.

According to some embodiments, system 100 may be configured to be used by firefighters or other emergency workers. In this case, system 100 may be particularly configured to assume a set shift schedule, but to adapt cases of the user sleeping on their shift, and for sleep to be disturbed, such as when a call-out occurs. System 100 may also be configured to deal with sleep that is only temporarily disturbed, such as when a call-out proves to be a false alarm and the user must attempt to fall back asleep immediately.

According to some embodiments, system 100 may be configured to be used by construction workers. In this case, system 100 may be particularly configured to assume a mixed schedule, and to further take into account the isolated environment in which many construction workers are located when providing recommendations.

According to some embodiments, system 100 may be configured to be used by mining workers. In this case, system 100 may be particularly configured to assume a fly-in, fly-out schedule, and to further take into account light exposure data based on the environmental context of the user.

According to some embodiments, system 100 may be configured to be used by defence workers. In this case, system 100 may be particularly configured to adapt to different shift structures, and to take into account the environment in which the user is operating, which may require system 100 to operate in an offline mode with low or no internet access.

According to some embodiments, system 100 may be configured to be used by heavy vehicle drivers or other long distance transport workers. In this case, system 100 may be particularly configured to assume that the user may be driving long hours with short breaks and sleep environments that are not optimal. System 100 may further be configured to take into account the isolated environment in which many transport workers are located when providing recommendations.

According to some embodiments, system 100 may be configured to be used by corporate managers and executives. In this case, system 100 may be particularly configured to assume that the user may be travelling across multiple time zones and working at variable times of day and night for short periods, which may cause their sleep schedule to be disrupted.

FIG. 3 illustrates a method 300 for identifying and reducing data infidelity for subjective and objective data received by device 110. In particular, method 300 identifies and reduces data infidelity of user responses to questions presented to them by device 110, specifically where the questions relate to shift work, sleep patterns, mood, alertness, and other lifestyle and wellbeing topics. Method 300 may be performed by processor 112 executing data capture and validation modules 160 during step 225 of method 200 as described above with reference to FIG. 2.

Infidelity in subjective answers to questionnaires is common due to user biases and misperceptions, which are particularly prevalent for users having sleep problems, as well as for older users. For example, insomnia can result in discrepancies in subjective responses relating to sleep when measured against objective data, due to altered patterns of brain activation associated with the insomnia disorder. Furthermore, sleep deprivation and lowered sleep quality, which are common in shift workers, are generally associated with cognitive deterioration that impairs the ability of the user to understand the questionnaire, and therefore lowers the fidelity of the answers.

The infidelity in responses to questionnaires can be overcome by asking redundant questions to confirm or validate the answers previously provided by the user, which can result in an improvement in the fidelity of the survey results. However, redundancy can also waste the time and energy of a user, and the user may become disinterested in the questionnaire if they feel they are being asked repeated questions. This issue can be mitigated by only asking redundant questions when a discrepancy exists between subjective answers and objective data, or when a discrepancy exists within multiple subjective answers about a particular metric.

Any redundant questions to be presented to a user should also be phrased in a way so that the intent of the question is understood by the user and so the user can provide an accurate response. If the intent of the redundant question is unclear, the responder could easily become confused, leading to a decrease in answer fidelity. The rewording and the order in which redundant questions are presented to a user must therefore also be determined to prevent the introduction of biases, such as order bias or halo effect bias.

Processor 112 performing method 300 by executing data capture and validation modules 160 may reduce answer infidelity in responses to an on-boarding questionnaire by integrating user's subjective answers with objective data, comparing subjective answers with objective data and/or further subjective data received in response to redundant questions, presenting the user with additional redundant questions only if inconsistency in the data is detected, and tracking trends to modify the wording of questions that consistently result in high infidelity. The additional redundant questions may be retrieved from question set 142, which may contain multiple paraphrased versions of each question that are generated at design time by a human operator. Alternatively, the redundant questions may be generated in real time by processor 112, which may be configured to execute automated sentence paraphrasing modules utilising natural language processing techniques to rewrite one or more questions while retaining the semantic meaning of the original question.

At step 301 of method 300, processor 112 executing objective data module 162 receives objective data, which could include data generated by device 110, or sensor data generated by sensor 159 and received from remote device 150. For example, objective data may include a time at which an alarm was set, whether a user snoozed their alarm, how frequently a user used device 110 or device 150, for what duration the user used device 110 or device 150, and/or a distance travelled by a user using device 110 or device 150, for example.

As objective data is not always accurate, processor 112 may further be configured to receive subjective data as a user confirmation of the accuracy of the objective data received at step 301. Furthermore, it may be desirable to determine whether there is a mismatch between subjective and objective data. At step 305, processor 112 executing subjective data module 163 receives subjective data, which may include user responses entered via user I/O 114 in response to questions or prompts presented by device 110. Subjective data may include the time the user reports going to sleep or waking up, how many times a user reports waking up during sleep, how much caffeine a user reports consuming, and/or a reported mood of the user, for example.

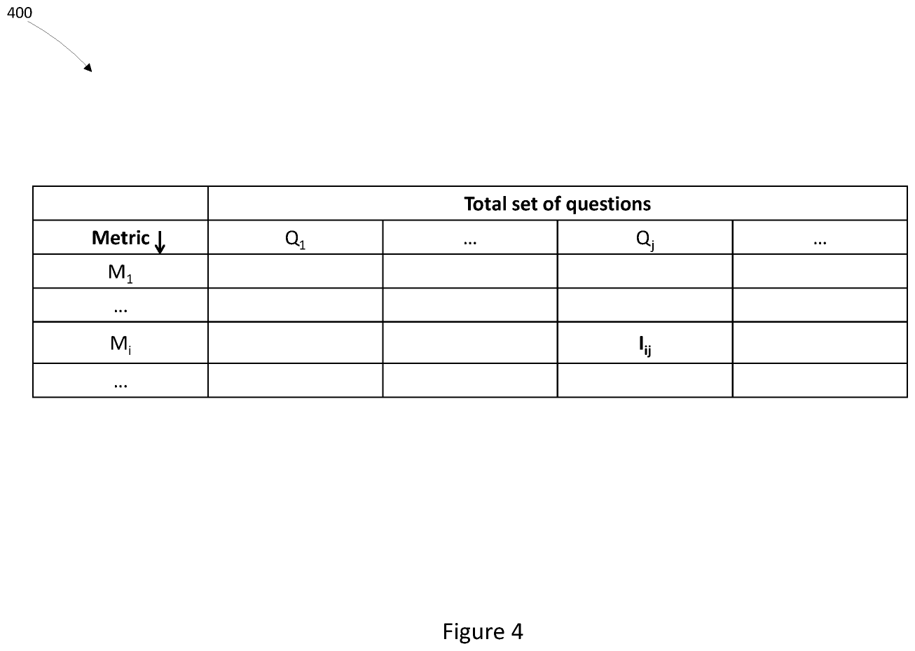

At step 310, processor 112 executing metric mapping module 164 processes the objective and subjective data to map each datum to one or more predetermined metrics retrieved from metric data 146 of database 140. For example, data may be processed to determine sleep metrics such as time in bed, total sleep time, wake after sleep onset (WASO), sleep onset latency (SOL), and sleep efficiency. This step is explained in further detail below with reference to FIG. 4.

At step 315, processor 112 executing data infidelity module 165 calculates an infidelity of the data for each retrieved metric. This calculation may be done by comparing data sets received for each metric, and the infidelity may be determined based on the degree to which data for a particular metric is inconsistent. Processor 112 may be configured to compare metrics that have been determined from subjective data with metrics that have been determined from objective data, and to determine whether mismatches exist. Mismatches having a value above a predetermined threshold may be determined to have a high data infidelity. The predetermined threshold value may vary across different metrics.

For example, where objective data generated by device 110 shows a user was using device 110 until 11 pm at night, but subjective data shows that a user reported going to sleep at 10 pm, processor 112 executing data infidelity module 165 may calculate a high data infidelity for the metric relating to the time at which the user fell asleep. In contrast, if subjective data shows a user reported going to sleep at 10 pm and waking up at 6 am, and further subjective data shows the user reported their sleep duration as being 8 hours, processor 112 executing data infidelity module 165 may calculate a low data infidelity for the metric relating to the duration of sleep of the user.

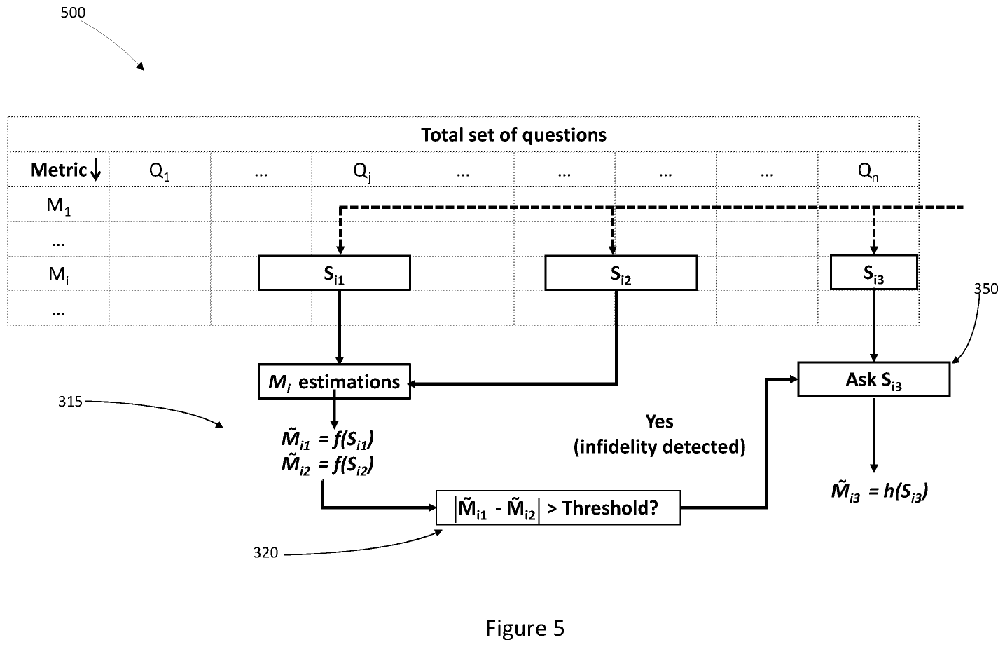

At step 320, processor 112 executing data infidelity module 165 determines whether the calculated infidelity exceeds a predetermined threshold for each metric, as described in further detail below with reference to FIG. 5. The threshold may be predetermined based on evidence-based known variations in metrics such as sleep duration, and adherence to prior sleep and wake recommendations. If processor 112 determines that the infidelity for a particular metric does not exceed the predetermined threshold, processor 112 may proceed to execute steps 325 and 330. At step 325, processor 112 may store a value for the metric based on the data associated with that metric in memory 120. Where there is a mismatch in metric values, processor 112 may determine the value for the metric to be the value that comes from the more reliable input source. A “data quality” or “data reliability” score for each input source may be used to determine this. If the accuracy of the input sources is unknown, processor 112 may determine the value to be the average of the metric values.

At step 330, processor 112 may pass the stored metric value as an input to recommendation generation module 170, which may use the value to determine at least one recommendation to present to a user, as described in further detail below with reference to FIGS. 8 to 12.

If at step 320 processor 112 determines that the infidelity for a particular metric does exceed the predetermined threshold, processor 112 executing data infidelity module 165 may proceed to execute step 335, by prompting the user to confirm the accuracy of the data relating to the metric. This may include the user confirming the accuracy of both subjective and objective data. For example, where objective data shows that device 110 was in use until 11 pm, but the user reports going to sleep at 10 pm, the user may indicate that the objective data is inaccurate due to a family member using device 110 at that time. Alternatively, the user may indicate that their subjective data was inaccurate, and may change this value to 11.30 pm upon seeing the conflicting objective data.

Once the user confirms the accuracy of the data, processor 112 executing data infidelity module 165 may check the infidelity again at step 340. If processor 112 determines that the infidelity for the particular metric no longer exceeds the predetermined threshold, processor 112 may proceed to execute steps 325 and 330 as described above. If processor 112 determines that the infidelity for a particular metric still exceeds the predetermined threshold, processor 112 may proceed to execute step 345.

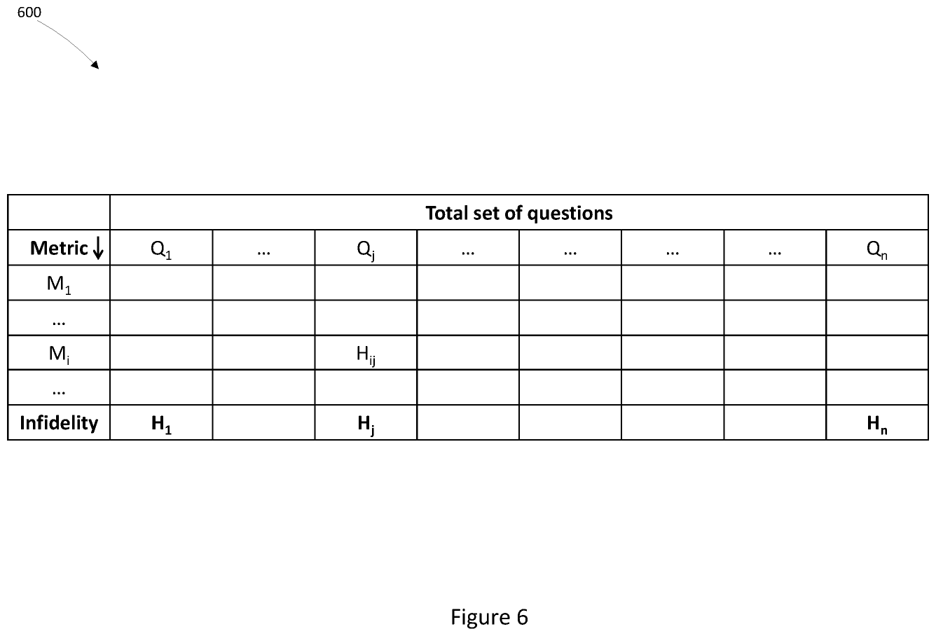

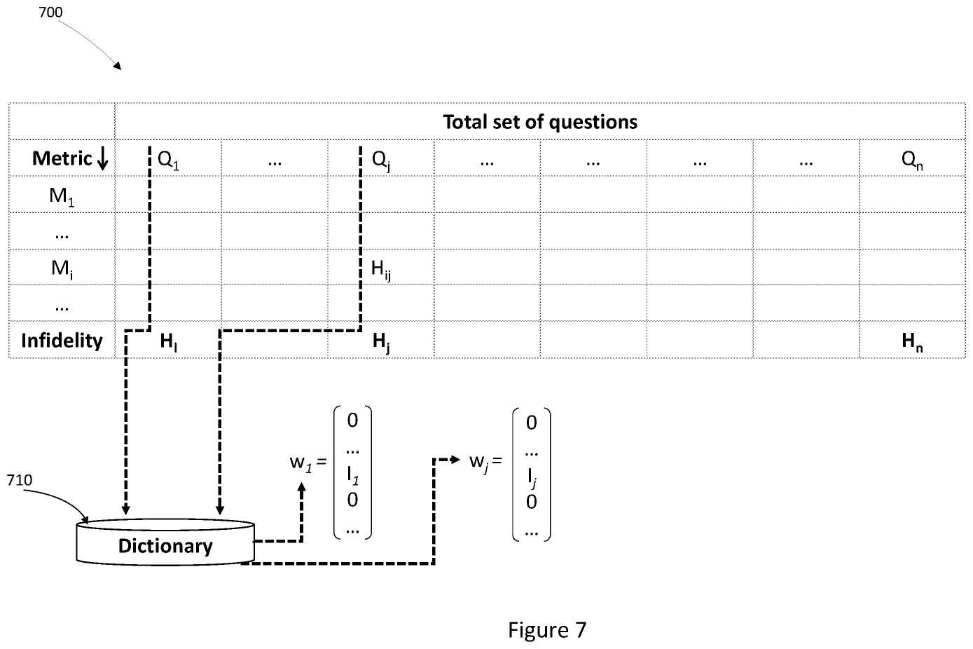

At step 345, processor 112 executing infidelity analysis module 167 stores any questions or prompts relating to the metric and the level of infidelity of the metric in memory 120. This may allow processor 112 to later identify questions, prompts and/or word combinations within the questions that cause confusion to the user, as described below with reference to FIGS. 6 and 7.

At step 350, processor 112 executing question generation module 166 prompts the user to provide further data associated with the metric. According to some embodiments, the user may provide further data in the way of objective sensor data generated by remote device 150. In some embodiments, processor 112 may generate further questions or retrieve further questions from question set 142 associated with the metric, and prompt the user to provide further subjective data by answering the question. Processor 112 then continues executing method 300 from step 315, by calculating a new infidelity for the metric based on the new data. Processor 112 may use the most current data to determine the metric value and the infidelity when executing step 315, and may discard the old data values.

Method 300 is described in further detail below with reference to FIGS. 4 to 7.

FIG. 4 shows a table 400 that may be generated by processor 112 executing metric mapping module 164. Table 400 lists a number of metrics M against a number of questions Q. Metrics M may be retrieved from metric data 146, while questions Q may be retrieved from questions set 142. Table 400 is used by processor 112 to map questions Q to metrics M. According to some embodiments, questions Q may also correspond to objective data, phrased as virtual questions such as “at what time did the user stop using device 150?”. The amount of information provided by a response to question Qⱼ on metric Mᵢ is referred to as Iᵢⱼ.

According to some embodiments, one metric may map to more than one question. According to some embodiments, one question may map to more than one metric. For example, a question directly asking about average sleep duration may correspond to a sleep duration metric M, and the combination of answers to questions “when do you go to sleep” and “when do you wake-up”, may also correspond to the sleep duration metric M. The first question constitutes a first subset, and the second two questions constitute a second subset informing the sleep duration metric. A third subset can contain the result(s) of objective methods to measure sleep duration. These three subsets are redundant because they inform a single metric. As objective measures are assumed to provide the highest amount of information, processor 112 may be configured to set the information value I corresponding to the objective questions relating to a particular metric as a high value. For example, where information values I may be set to any values between 0 and 1, an information value I corresponding to an objective question for a particular metric may be set to 1. Information value I may be used to weight the data, particularly when the data is received from multiple data sources relating to a single metric.

Where multiple questions Q relating to a single metric M provide substantially different responses, processor 112 may determine that these responses have a high infidelity, and that further information is required to determine the true value of the metric. This may require prompting the user to verify the objective or subjective data, or prompting the user to enter additional data.

This is illustrated by table 500 as shown in FIG. 5, which shows metrics M mapped to questions Q, along with select method steps from method 300. Information values Sᵢ₁, Sᵢ₂ and Sᵢ₃ correspond to responses to questions Q that map to metric Mᵢ. The questions relating to these values are therefore redundant. Processor 112 may start by asking the questions relating to Sᵢ₁ and Sᵢ₂. The metric estimations for metric Mᵢ are determined by processor 112 at step 315, with the first value estimate Mᵢ₁ being the function of response Sᵢ₁, and the second value estimate Mᵢ₂ being the function of response Sᵢ₂. At step 320, processor 112 determines whether the absolute difference between the metric value estimates exceeds a predetermined threshold. If the threshold is exceeded, then at step 350 processor 112 prompts the user for further information, by asking the questions relating to information Sᵢ₃. A third metric estimate Mᵢ₃ is then calculated by processor 112 based on information Sᵢ₃.

Where processor 112 is configured to monitor sleep maintenance insomnia due to shift work performed by a user, processor 112 requires data relating to the number of times a user wakes up within a sleep session. Processor 112 may be configured to initially present a user with questions retrieved from questions set 142 to directly ask the user to subjectively report the average number of times they wake-up within a typical sleep session. Processor 112 may also ask the user to submit objective data including the clock times at which they wake-up. The objective data may be used to verify the fidelity of the subjective data. For example, if the patient reports waking-up three times and the objective data reports a single time, the patient may be asked to check their answer to ensure higher accuracy of the data.

In accordance with some embodiments, processor 112 may also execute infidelity analysis module 167 to track and analyse answer infidelity, as described above with respect to step 345 of method 300 and as described in further detail below with reference to FIGS. 6 and 7. This may allow processor 112 to identify particular sets of questions Q that consistently produce answer infidelity above the predetermined threshold. This log of questions can be used to identify questions with ambiguous wording and unclear intents. According to some embodiments, processor 112 executing infidelity analysis module 167 may be configured to track and log questions that result in infidelity in a predetermined percentage of users. For example, according to some embodiments, processor 112 executing infidelity analysis module 167 may be configured to track and log questions that result in infidelity in at least 20% of users.

Once these questions are logged by processor 112, the questions can be updated to improve clarity and thus decrease the amount of answer infidelity. Over time, rewording questions or providing additional information to clarify questions may lead to a decrease in the number of confusing questions and, ultimately, to convergence of a list of optimally worded questions with clear intents. Clearer questions may result in more accurate information from users, which will ultimately result in an increased success rate with recommendations generated by processor 112 executing recommendation generation module 170.

FIG. 6 shows a table 600 having questions Q mapped to metrics M. Table 600 also maps a marginal infidelity value H to each question, based on infidelities Hᵢⱼ determined for each question and metric pair Qⱼ and Mᵢ across a user population, or for a particular section of the user population (such as for elderly users, for example). Processor 112 may be configured to integrate marginal infidelities H down each column corresponding to each question Q to estimate the infidelity H associated with each question.

FIG. 7 further illustrates using a similar process to identify the infidelity for particular word combinations found within the questions. FIG. 7 shows a table 700 having metrics M mapped against questions Q, with each question and metric pair having an infidelity value H, which is integrated down each column to calculate an infidelity for each question Q. FIG. 7 further illustrates processor 112 projecting questions into a word space defined by a dictionary 710 containing V most common words. Such projection leads to sparse vectors w₁ and w₂, having V components. The entries of vectors w₁ and w₂ are equal to 1 only at positions corresponding to words present in a given question. Processor 112 can therefore determine whether a particular word-combination pair is likely to result in infidelity-inducing questions.

For example, dictionary 710 may contain the elements: {‘Sleep time’, ‘Duration’, ‘How long’, ‘Sleep’ }. If a first question Q1 was “What was the duration of your sleep?”, this question may be represented by the vector: {0, 1, 0, 1}. If a second question Q2 was “How long did you sleep?”, this question may be represented by the vector: {0, 0, 1, 1}. If the level of infidelity or error when asking question Q1 was 60 minutes, and the infidelity or error when asking question Q2 was 20 minutes, processor 112 may determine that vector {0, 1, 0, 1} is associated with an infidelity of 60 and that vector {0, 0, 1, 1} is associated with an infidelity 20, for example.

FIG. 8 relates to a method 800 for prioritizing the order of sleep improvement recommendations provided to a user by system 100, as described briefly above with reference to step 235 of method 200.

The profiles of shift workers can vary significantly from one to another, since the definition of a shift worker can include individuals with very different work schedules, work environments, socio-economic backgrounds, demographics, physical traits and attitudes. This makes it particularly challenging to provide a one-size-fits-all solution to sleep pattern management for shift workers. Recommendations to improve sleep patterns in shift workers need to be tailored to them to facilitate the best outcomes.

The effectiveness of any recommendations may depend on the individual user's response to the recommendation, the user's adherence to the recommendation, the user's perceived and objective success in resolving their identified problem(s) based on the recommendation, and on any changes to the subjective reporting by the user of previously reported values.

It is important for the user to pick the recommendation that is most likely to be effective for their situation, schedule and habits. The order in which a list of recommendations is communicated to a user can therefore affect the treatment success, consumer satisfaction, and consumer adherence to a recommendation. Arbitrarily selecting the order in which the recommendations are presented (either visually, orally, or a combination thereof) has disadvantages including that the user is often left to guess which recommendation to address first. Furthermore, the likelihood of picking a less effective treatment is higher, which might result in decreased compliance or even complete abandonment of a treatment program. Selecting a single, static ordering of recommendations to present to a user based on aggregate or population statistics fails to address the lifestyle and profile differences from one shift worker to another.

Method 800 when performed by processor 112 executing recommendation delivery modules 180 causes processor 112 to utilise the machine learning technique of clustering to assign each user profile to a user cluster. Recommendations presented to the user are then iteratively improved by prioritising the recommendations based on effectiveness of the recommendation for the specific user cluster. This prioritisation allows the most effective recommendation to be presented first to the user when more than one recommendation is to be communicated, or if the recommendation involves multiple steps or parts.

Method 800 starts at step 805, where processor 112 executing user profile data module 161 receives user profile data as described above. At step 810, processor 112 executing objective data module 162 receives objective sensor data, and at step 815 processor 112 executing subjective data module 163 receives subjective response data. The data may relate to the user's line of work, work schedule, sleep routine and habits, demographic and health information.

At step 820, processor 112 executing clustering module 181 uses the user profile, objective data and subjective data of all users in the system 100 to determine user clusters for the user population, and to identify the cluster for the particular user for which recommendations are to be generated. Data for the user population may be retrieved from user profile data 144 of database 140. The user data for the user of device 150 may be pre-processed into a normalized data vector by processor 112 using one of several appropriate normalization techniques, which may include min-max normalisation, which involves fitting the data vectors into pre-defined boundaries, for example. Another appropriate technique may be the elimination of outliers. Processor 112 then applies logical and unsupervised machine learning techniques, which may include agglomerative clustering techniques, to the data in order to find the participant cluster cⱼÎ C which minimizes an arbitrary distance or cost function between the user data vector, and the cluster centre of that cluster. Clustering techniques, such as partitioning clustering, k-means clustering and hierarchical clustering may be used. The choice of clustering technique may be based on the specific metrics and the data collected, to provide the most distinct user groups or clusters, which may be determined through experimentation.

Database 140 may maintain an ongoing partitioning of participants into a set C of n_{c} (D(t),nᵣ) clusters, with D(t) representing the state of all participant data at some point in time (t), and nᵣ representing the number of possible unique recommendations. The number of clusters n_{c} ((D(t),nᵣ) may be limited by clustering best practices, and may grow or shrink as a function of available data quantities and population similarity measures discovered.

Processor 112 finds the optimal recommendation list for each cluster, being one unique set of ordered recommendations, defined as one permutation sᵣ Î Sᵣ of ordered recommendations, where the size of Sᵣ can be computed as the number of arrangements of recommendations (noting that participants may receive up to nᵣ recommendations), defined as

$$a ( {n}_{r} ) = {\sum }_{k = 0}^{{n}_{r}} ( \begin{matrix}n \\ k\end{matrix} ) k !$$

At step 825, recommendations are generated by processor 112 executing recommendation generation module 170. Recommendations may include sleep hygiene tips and prescriptions or references to products/devices that can help improve the wellbeing of the shift worker.

At step 830, processor 112 executing recommendation ordering module 182 determines the order of the generated recommendations to present to the user based on the cluster to which the user belongs. The ordered set of recommendations s_{c,i} found to be optimal for the cluster is used as the initially prioritized set for the user. Processor 112 executing recommendation ordering module 182 may act as a recommendation engine to determine which of the nᵣ recommendations are irrelevant or infeasible to the user, and may eliminate these from the user's set. For example, a recommendation to “Avoid walking pets right before attempting to sleep” may be irrelevant to a participant who has no pets, but may be very effective for a given cluster cᵢÎ C. This elimination may be considered to be a recommendation masking step in order to avoid providing irrelevant or infeasible suggestions. If at least one recommendation is masked, processor 112 may further be configured to provide an alternative recommendation to replace the at least one masked recommendation.

Processor 112 then executes recommendation presentation module 183 to present the recommendations to the user in the determined order via user I/O 114. According to some embodiments, all of the generated recommendations may be presented to the user simultaneously. According to some alternative embodiments, the generated recommendations may be presented to the user one at a time. A subsequent recommendation may be presented to a user only once a previous recommendation has been implemented or attempted, for example.

According to some embodiments, processor 112 executing recommendation presentation module 183 may also use social influence principles when communicating the recommendations, to explicitly highlight the degree of effectiveness in treating similar users for each particular recommendation. For example, as shown in FIG. 9, each recommendation may be displayed with a number of patients for whom the recommendation was useful. FIG. 9 shows a table 900 having a number of recommendations 910 and a number of corresponding impacts 920. For example, table 900 shows that for the recommendation “Keep a regular bedtime schedule (with variability shorter than 1 hour)”, the corresponding impact is “This was effective in XX % of patients with similar characteristics”. Seeing a high impact in similar users may encourage users to attempt the particular recommendation.

Returning to FIG. 8, at step 835 processor 112 executing subjective data module 163 may generate and present a questionnaire to the user to gauge the effectiveness of the recommendations. This may be done after a period of time has elapsed after providing the user with the recommendation. According to some embodiments, a new questionnaire may be presented to the user periodically to gauge the effectiveness of the recommendation they have decided to use. According to some embodiments, processor 112 may further execute objective data module 162, and further objective data may also be received.

At step 840, processor 112 updates the recommendation effectiveness data stored in database 140 based on the responses received from the user at step 835, and the process is iteratively repeated. The recommendation effectiveness data may then be used to provide recommendations to future users with similar user profiles and sleep schedules, for example.

The cadence of the iterative process can be accelerated by shortening questionnaires presented to the user, such that only most relevant questions for the treatment are included. The most relevant techniques may be identified based on the user profile data and the user cluster, for example. Cadence can also be accelerated by increasing the frequency of questionnaire presentation, for example by increasing it to daily instead of weekly. If a large amount of user data is available in database 140, the convergence to a list of optimally ordered recommendations can be accelerated.

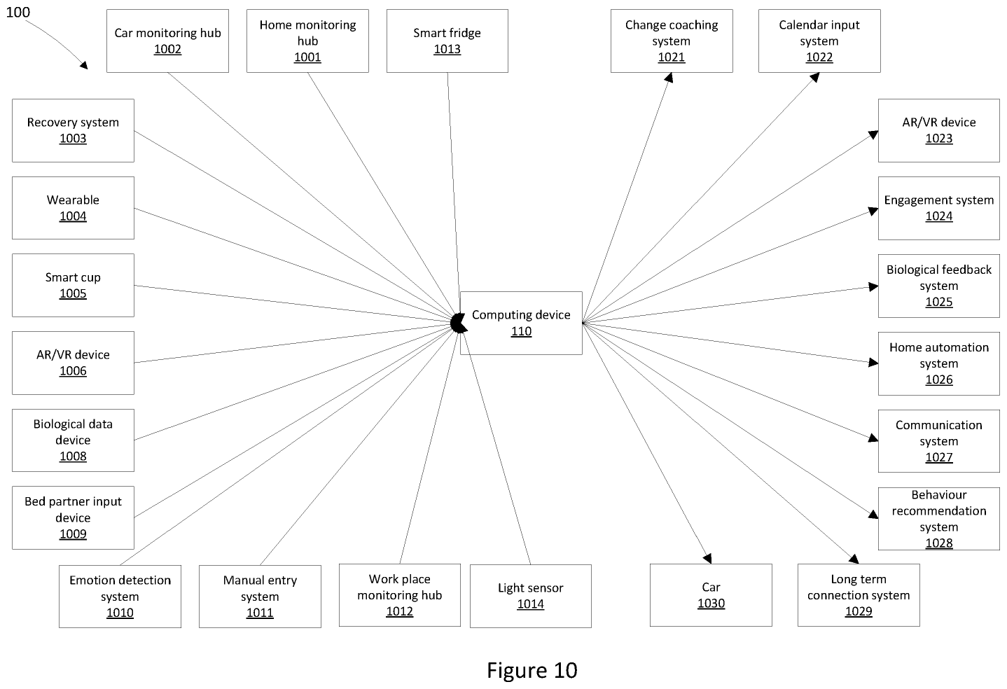

FIGS. 10 to 12 describe the functions performed by processor 112 executing modules 160, 170 and 180 in further detail.

FIG. 10 is a block diagram illustrating a number of input and output systems that may provide data to and receive instructions from processor 112 executing modules 160, 170 and 180. In particular, FIG. 10 illustrates a number of forms that may be adopted by remote device 150, which may be in the form of devices or systems. Specifically, device 150 may include one or more of devices and systems 1001 to 1014 or 1021 to 1030.

Devices and systems operating to generate input data for device 110 may include home monitoring hub 1001, car monitoring hub 1002, recovery system 1003, wearable 1004, smart cup 1005, augmented reality or virtual reality device 1006, biological data device 1008, bed partner input device 1009, emotion detection system 1010, manual entry system 1011, work place monitoring hub 1012, fridge 1013 and light sensor 1014, for example.

Devices and systems operating to receive instruction data from device 110 may include change coaching system 1021, calendar input system 1022, augmented reality or virtual reality device 1023, engagement system 1024, biological feedback system 1025, home automation system 1026, communication system 1027, behaviour recommendation system 1028, long term connection system 1029, and car 1030, for example.

Home monitoring hub 1001 may be located in the home of a user, and may include environmental sensors, and home assistance features. For example, home monitoring hub 1001 may include environmental sensors that allow home monitoring hub 1001 to monitor humidity, a location of a user, light levels, temperature, volume within the home, and the user's schedule, for example. Home monitoring hub 1001 may also monitor home assistance features to determine a user's sentiment, such as mood and stress levels; to detect conflicts such as stress of the primary user, their spouse or family; to detect the sleep and wake times of the user, to keep track of lists such as to-do lists and shopping lists, and to monitor activities discussed in the home.

Car monitoring hub 1002 may be located in the car of the user, and may be configured to monitor the speed, swerving, music and temperature in the car. According to some embodiments, car monitoring hub 1002 may also monitor a user's blink rate or eyelid closure (for instance using computer vison methods), to determine their tiredness levels.

Recovery system 1003 may be configured to assist a user in recovering from a poor sleeping pattern. Recovery system 1003 may comprise a number of devices, and may be configured to monitor sleep duration, sleep time, light and caffeine intake by a user.

Wearable 1004 may be a smart watch or other wearable computing device configured to be worn by a user. Wearable 1004 may comprise an electroencephalogram (EEG) in some embodiments. Wearable 1004 may be configured to monitor sleep staging, exercise and activity, heart rate, awakenings during the night, time in bed, time awake, a galvanic skin response indicative of mood or emotion, environmental light levels, and the location of the user, which may be done via a GPS module.

Smart cup 1005 may be a drinking vessel configured to monitor caffeine, alcohol and sugar consumed by a user via the vessel.

Augmented reality or virtual reality device 1006 may be configured to monitor the augmented reality and virtual reality activity by the user, which may include community or group communication, communication with a therapist, and a user's blink rate.

Biological data device 1008 may be configured to monitor biological data such as weight, stress, nutrition, medications and anxiety of a user.

Bed partner input device 1009 may be configured to monitor a bed partner of a user, which may include monitoring the biological information of their bed partner, the mood of their bed partner, and the bed partner's rating of the user's sleep.

Emotion detection system 1010 may monitor an emotional state of the user, such as how they are feeling and how they are coping.

Manual entry system 1011 may allow a user to manually enter information, such as their shifts, mood, caffeine intake, light levels, sleep, nutrition, exercise levels, and alertness levels, for example.

Work place monitoring hub 1012 may be located in a work place of the user, and may be configured to monitor a workplace of the user. For example, work place monitoring hub 1012 may monitor the number of stressful minutes the user experienced over a shift at work, the user's sentiment including mood and emotion, and the user's shift calendar.

Fridge 1013 may be a smart fridge, and may be configured to monitor grocery purchases, scan grocery receipts, save grocery orders, and monitor nutrition information of food items placed in or removed from fridge 1013.

Light sensor 1014 may be configured to monitor light exposure to the user, and may form part of a wearable device in some embodiments.

Change coaching system 1021 may receive instructions from device 110, and implement changes to the user environment, which may include changes to their bedroom via home automation, advertising products to the user to help them implement recommendations, providing sleep hygiene coaching, ensuring compliance of the user with therapy, providing stress management techniques such as meditation, progressive muscle relaxation and exercise, and providing nutritional recommendations, such as a circadian diet, a third party diet program, or a home cooking delivery service.

Calendar input system 1022 may receive instructions from device 110, and automatically input entries into a calendar of the user. For example, calendar input system 1022 may add entries for additional activities, recommended changes in schedule, shift swapping, scheduled sleep or naps, identifying ways other shift workers have optimised their time, and suggesting services that may free up time, such as cooking, cleaning and shopping services.

Augmented reality or virtual reality device 1023 may receive instructions from device 110, and may provide services to the user such as guided meditation, progressive muscle relaxation, suggestions to wind down while watching television, playing soothing music, and releasing soothing scents into a bedroom of a user, for example.

Engagement system 1024 may receive instructions from device 110, and provide the user with engagement such as links to articles, links to their coach, links with their therapist, routes to a support group, and gaming options, for example.

Biological feedback system 1025 may receive instructions from device 110, and may provide recommendations to the user regarding the user's stress and anxiety, and suggestions for meditation.

Home automation system 1026 may receive instructions from device 110, and may act on those instructions to alter the temperature, lighting, bed softness, and sounds such as music, podcasts or audio books in the user's home.

Communication system 1027 may receive instructions from device 110, and facilitate communication between the user and their therapist, community, bed partner, house mate, or program buddy.

Behaviour recommendation system 1028 may receive instructions from device 110, and provide recommendations regarding when the user should sleep, eat, and meditate, for example.

Long term connection system 1029 may receive instructions from device 110, and may facilitate the user drawing connections over time with respect to their schedule, habits, people they work with, recovery score, calendar activities, stressful environments, work place stress, and mood.

Car 1030 may receive instructions from device 110, and may act on those instructions to alter the temperature, seat comfort, and audio in the user's car.

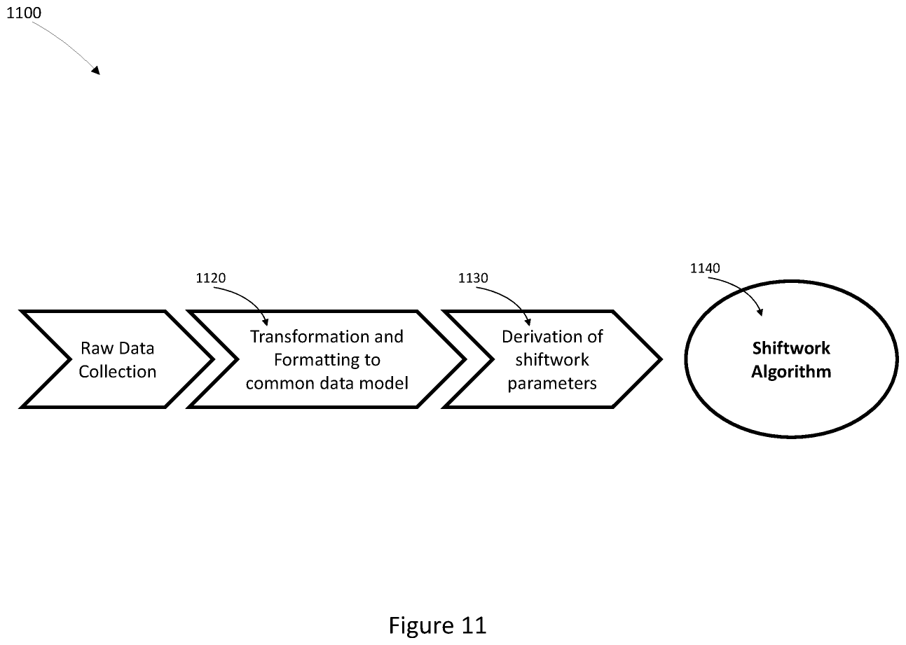

FIG. 11 shows a block diagram 1100 illustrating how data may be processed by processor 112 after being received from input devices and systems 1001 to 1014. At step 1110, processor 112 received the raw data from the device or system, which may be raw sensor data generated by a sensor such as sensor 159. As described above with reference to FIG. 10, the raw data may include data from home or car monitoring, wearables, smart devices such as smart cups, augmented reality data, biological data, bed partner data, self-reported data, and workplace data, such as shift times and performance.

At step 1120, processor 112 transforms and formats the raw data to a common data model. Since the raw data comes from various sources, the format of the raw data may vary. For example, objective data from a wearable device such as a smart watch may be in JSON format. Data from another wearable device may be output in the form of a CSV file. Processor 112 may translate the data of various forms into a single data type and/or format to allow the data to be readily compared.

At step 1130, processor 112 then derives a number of shift work parameters from the data, which may include circadian cycle, alertness, sleep debt, sleep hygiene, adherence and health and wellness parameters.

At step 1140, processor 112 then executes modules 160, 170 and 180 to perform a shift work management process to generate recommendations and implementations for the user. The shift work management process may include using a decision tree informed by best practice circadian principles in some embodiments. According to some embodiments, the shift work management process may include using a model driven recommendations model, which may be a bio-mathematical or biophysical model in some cases. According to some embodiments, the recommendations model may be a model as described in WO/2013/110118, the entirety of which is herein incorporated by reference. According to some embodiments, the shift work management process may predict alertness, sleep, and circadian dynamics under a variety of conditions, including normal daytime activities, shift work, and jetlag. The shift work management process may use a system of ordinary differential equations, which may be developed based on knowledge of neurobiological mechanisms of sleep and circadian regulation. According to some embodiments, the shift work management process may be calibrated to generate recommendations for a standard individual or group average.

According to some embodiments, the shift work management process may be personalized for individuals by adjusting model parameters.

An example model for use by the shift work management process may use input data including shift times, work times and wake up times; light and dark cycle information, including light levels at a workplace and light levels at home during sleep; constraints such as times when sleep cannot be recommended; caffeine intake and chronotypes, for example. The shift work management process may generate outputs such as an alertness level; a level of sleep and sleepiness; circadian phase estimates including dim light melatonin onset (DLMO); and caffeine and light exposure or avoidance. The outputs of the shift work management process may include predictions and/or recommendations that correspond to the biological dynamics of an average or typical person.

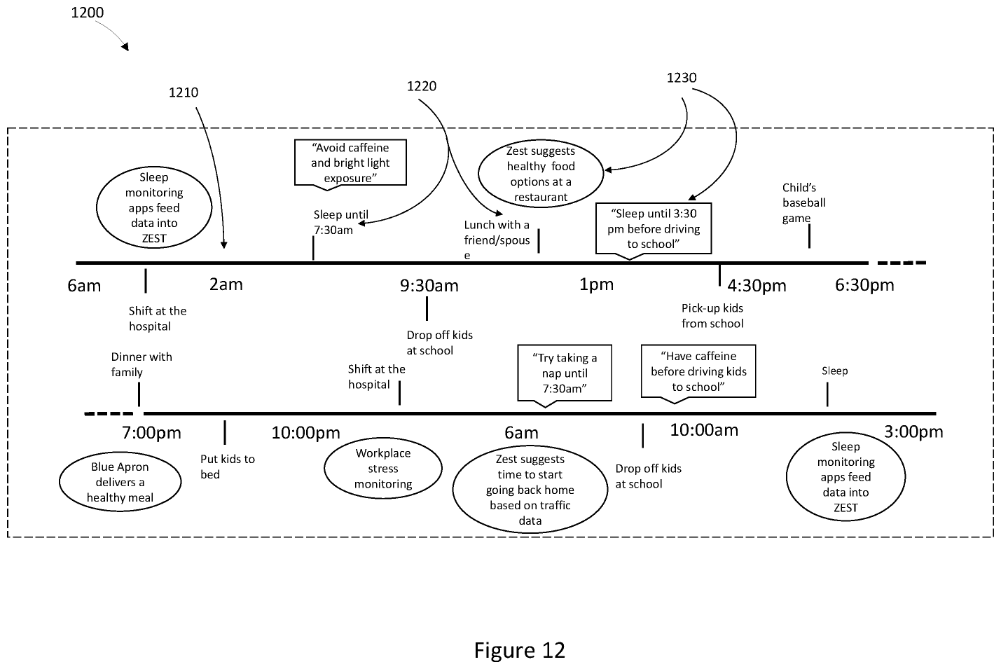

FIG. 12 illustrates an example timeline 1200 having an axis 1210 showing times in a user's day. Timeline 1200 shows example activities and actions 1220 scheduled or performed by a user, as well as recommendations 1230 generated by system 100. For example, activities 1220 include a shift at the hospital, sleeping until 7.30 am, dropping the kids off at school, and having lunch with a friend or spouse. System 100 makes recommendations 1230 such as participating in workplace stress monitoring, having sleep monitoring apps active, avoiding caffeine and bright light exposure, and suggesting healthy food options at a restaurant.

System 100 could provide a number of functions to assist a user in managing sleep and shift work. For example, according to some embodiments, system 100 may provide circadian rhythm monitoring.

Light information from a user's immediate environment, including duration of exposure, intensity and spectral composition affects the nature of the user's body's response to light. Extended exposure to blue-enriched light at irregular times may disrupt the homeostatic process of the user's body. Bright light exposure affects the physiological parameters like sleep quality, mental performance and daytime alertness among others. Sleep quality and mental health can be improved by controlling the duration of exposure and spectral parameters of light.

Where sensor 159 of remote device 150 comprises a light sensor, processor 112 executing recommendation generation module 170 could be configured to provide insights on the amount of light exposure that is appropriate at a particular time of the day/night, depending on the user's work and sleep schedule. For instance, if the user had a work shift that ended at lam, processor 112 executing recommendation generation module 170 may recommend that the user avoid bright light exposure to be able to sleep faster. This may include limiting screen time and dimming room lights, for example. Processor 112 executing recommendation generation module 170 may also suggest ideal times to sleep, nap and be awake depending on the light exposure, and on shift schedules that may be either manually or automatically input.

Some shift workers have limited exposure to light by nature of their work, such as shift workers who work in mining. In contrast, some shift workers, such as those working in hospitals or at desk jobs involving computers, might have excessive bright light exposure during shift times. Processor 112 executing recommendation generation module 170 may be configured to take into account typical light exposures depending on the nature of the user's work, their shift schedules, commute time to and from work, and location and weather, and may be configured to suggest optimal bedtimes. Processor 112 executing recommendation generation module 170 may also suggest that the user have exposure to natural light in the event that there is no light exposure detected by sensor 159 for an extended period during the day. For example, processor 112 executing recommendation generation module 170 may suggest that the user increases exposure to bright light on particularly gloomy days, such as by taking a walk outside, to prevent the user being in a state of lowered alertness and mood.

Where sensor 159 comprises EEG, ECG, PPG, actigraphy, or other sleep monitoring modules, processor 112 may be configured to determine a user's sleep onset latency (SOL), sleep architecture, time in bed (TIB), total sleep time (TST) and number and duration of awakenings, among others. Processor 112 executing recommendation generation module 170 may use this data to generate recommendations around light exposure before and after bedtime to ensure optimal, relaxing sleep sessions.

Shift workers may struggle to keep up with their family/children's schedules and may miss out on important family events. Processor 112 executing recommendation generation module 170 may support the social life of the user syncing their work and sleep schedules with that of their partner/spouse and/or children. For example, if the user got off work at 6 am, processor 112 executing recommendation generation module 170 may suggest that they stay awake by exposing themselves to bright light, so that they can drop off their kids to school at 9 am.

According to some embodiments, system 100 may also provide recommendations relating to sleep debt.

Where sensor 159 comprises EEG, ECG, or PPG modules, or other modules configured to generate sleep data, processor 112 may be configured to extract parameters such as TST, SOL, TIB, time awake, sleep architecture, and number and duration of awakenings from the data generated by sensor 159.

Based on these parameters, shift schedules, family calendars, work location and commute time, processor 112 executing recommendation generation module 170 may recommend optimal sleep, nap, and wake times. Processor 112 executing recommendation generation module 170 may also suggest that a user avoid certain activities such as driving back after work if the user has a sleep debt from the previous sleep session or had a disturbed sleep session with increased number of awakenings. If the user is taking public transport to commute to and from work, processor 112 executing recommendation generation module 170 may recommend that commute time would be a good opportunity to catch a nap before or after a shift. Processor 112 executing recommendation generation module 170 may also suggest playing uplifting music before a shift to make the user feel more energized and ready for work.

If the user has had a disturbed sleep session prior to a shift either due to his/her own stress affecting sleep quality or due to their partner or spouse having a sleep disorder such as snoring, sleep apnea or restless leg syndrome, processor 112 executing recommendation generation module 170 may recommend that the user avoid doing things that need their full attention and find time to take small naps, to help the user feel more driven.

According to some embodiments, system 100 may also provide recommendations relating to health and wellness.

The overall health and wellness of a user may be dependent on both their physical and mental health. Due to the demanding hours and nature of shift work, shift workers may experience adverse physiological and psychological effects, including cardiovascular disease, depression, and anxiety. Proper nutrition, physical activity, and sleep may help alleviate these significant health risks.

Where sensor 159 is configured to generate physiological data regarding the user's overall wellness, processor 112 executing recommendation generation module 170 may recommend appropriate actions for the user to perform to improve their health. Metrics such as heart rate variability (HRV) and galvanic skin response (GSR) may be used in addition to sentiment analysis with a home assistant to determine a user's mood or stress level. Coaching could be provided by the processor 112 prior to or after a shift based on the inputs and subjective data given by the user on perceived mood and stress. According to some embodiments, processor 112 executing recommendation generation module 170 may make recommendations based on the user's age, gender, and previous health issues if the user is willing to share details such as mental disorders, sleep disorders, and prior injuries. As a result, the user may be able to improve their performance at work by taking the proper action outside of work to better their health.

With a constantly changing work schedule, shift workers have limited time to prepare nutritious and well-balanced meals. If the user makes grocery lists on device 110 or device 150, processor 112 executing recommendation generation module 170 may provide recommendations of food for the user to buy based on the user's preferences from previous lists and nutritional information. Processor 112 executing recommendation generation module 170 may also communicate with food delivery services like Home Chef or Blue Apron to assist in planning and preparing healthy meals for the user, taking into account the time the user has available to prepare and cook them. Additionally, processor 112 executing recommendation generation module 170 may make recommendations regarding when the user should eat to optimize sleep and recovery around their shift schedule. Connected home devices such as a smart refrigerator may be configured to monitor what the user eats, smart scales may be configured to monitor the weight or body mass index (BMI) of the user, and a smart cup may be configured to monitor caffeine and alcohol intake by the user. This data could be used by processor 112 executing recommendation generation module 170 to make diet and lifestyle change recommendations, or to suggest a coach, therapist, or dietician that is best suited for the shift worker's schedule.

According to some embodiments, system 100 may also provide recommendations relating to a user's degree of wakefulness or sleepiness.

Excessive fatigue is prevalent with shift workers, as long and late hours disrupt the person's natural sleep and recovery time. Furthermore, these work times can lead to chronic sleep deprivation and poor sleep quality, which reduces the worker's attention, cognition, and motor skills. This can increase the risk of accidents at work and adversely affect the worker's performance. Outside of work, excessive fatigue can affect the individual's ability to drive and negatively impact their social interactions with family and friends.

To address shift worker's fatigue, processor 112 executing recommendation generation module 170 may provide coaching for the user to sleep at the calculated optimum times for recovery. To characterize the shift worker's sleep quality, device 110 may integrate with sleep monitoring products, such as wearable monitors. Through these products, input regarding SOL, wake after sleep onset (WASO), TST and TIB can be quantified objectively and subjectively and used by processor 112 to tailor coaching to the user's needs. Additionally, input from the user's bed partner can be used to improve the recommendations from processor 112 executing recommendation generation module 170, for issues that the user may not be aware of including snoring, sleep apnoea and restless leg syndrome.

Since many shift workers' commute home occurs after a long overnight shift, the individual's alertness is imperative to their safety and others on the road. Based on wearable feedback worn by the user, or eye blink detection via the windshield of the user's car, processor 112 executing recommendation generation module 170 may determine the shift workers alertness level and be configured to play energizing music and turn down the temperature if processor 112 determines that the user may be falling asleep. Alternatively, if the user is over stimulated from a difficult shift, processor 112 executing recommendation generation module 170 may cause the user's car to play soothing music, change the low lights in the car to a pleasing colour, and gently prepare the user to wind-down from work. Furthermore, processor 112 may communicate with insurance apps that track the user's behaviour while on the road, to gain more inputs regarding the user's wakefulness.

Monitoring other inputs such as caffeine intake and medications that the user may use to stay awake or fall asleep may assist processor 112 executing recommendation generation module 170 to provide more relevant recommendations for the user. Additionally, processor 112 executing recommendation generation module 170 may use built-in screen-time trackers in devices 110 and 150 to alert the user if they are taking in too much blue light before sleeping.

According to some embodiments, system 100 may also provide recommendations relating to stress management.

For many shift work occupations, the nature of the work alone is stressful. Compounding this with a demanding work schedule, shift workers may struggle to manage their stress levels during and outside of work. Stress may not only affect the health of these workers, but may also negatively impact their job performance and social life.

Where remote device 150 is a car monitoring device, processor 112 may use car monitoring and feedback to help a user manage stress on their way to work or on their way home. For example, if the user is on their way to work, processor 112 executing recommendation generation module 170 may constantly update traffic information according to the shift worker's schedule to optimize their commute time. Processor 112 executing recommendation generation module 170 may also make recommendations to the user regarding their clothing based on weather data, to ensure the user is properly dressed and prepared for weather at the beginning and end of their shift. During the user's commute, remote device 150 may monitor the user's stress level, or ask the user questions about how they are feeling. Additionally, processor 112 may cause the car audio to update the user about what to expect when they get to work by pulling data from a workplace monitoring hub that monitors workplace stress and overall mood at the user's workplace. Based on what the user will expect at work, their mood, and ability to cope in the moment, processor 112 executing recommendation generation module 170 could provide talk therapy, soothing or energizing music, or adjust the temperature in the car.

Since shift workers are always on the go, they can struggle to keep track of their busy schedule, balancing family, work, and social activities. Processor 112 may be in communication with smart speakers, which could be used to input or change schedule information, add events to the user's calendar, or display calendars on a visual version of the smart speaker. The smart speakers could also listen for events being discussed by family members in the home or co-workers in the office, to provide data to processor 112, which could generate suggestions for adding events to the calendar. Processor 112 executing recommendation generation module 170 may analyse the user's to-do lists and shopping lists, and recommend times to complete these activities or recommend ways to complete the activities without spending time on them by leveraging services like online shopping and delivery, grocery delivery, cleaning services. This would enable the user to spend time on the things they are passionate about and with loved ones.

Using a combination of inputs including physiological stress response data and alertness, shift schedule, and current relaxation techniques that the shift worker uses, processor 112 executing recommendation generation module 170 may prompt the user to start a wind down routine in which the environment in the user's home may change to help the user wind down prior to sleep. Processor 112 executing recommendation generation module 170 may cause the lights to dim, the thermostat to be tuned up or down, soothing music to be played, or guided meditation or progressive muscle relaxation exercises to be initiated. These could be delivered via video, voice, or AR/VR headsets.

Processor 112 executing recommendation generation module 170 may automatically pull inputs from the user's shift work roster to display their shifts on their calendar. Processor 112 executing recommendation generation module 170 may also identify shifts of colleagues that would work better in the user's schedule as well as in the colleague's, and make suggestions for swapping shifts. This may allow the user to automatically balance family and children obligations with their work schedule, reducing the user's stress regarding these tasks.

According to some embodiments, system 100 may also provide recommendations relating to adherence to coaching.

Processor 112 may be configured to track adherence to the recommendations and coaching provided to the user, and to any improvement in the sleep, alertness and mood of the user during daytime activities. Processor 112 may gauge adherence using information such as the different sleep parameters (SOL, TST, TIB, sleep architecture, time awake, number and duration of awakenings), conflicts in the home (indicative of stress management capabilities), attendance in family events, alertness levels at work and family events, and mood.

Processor 112 may learn from the user's mood and behaviour over time, and tailor the coaching and recommendations provided to the user appropriately. For example, the rate at which a to-do list is checked off gives an idea of how the user is managing their daily activities. Over time, if the to-do list is not checked off, device 110 may prompt the user to reschedule some of the items on their list and work towards a more organized and relaxed to-do list.

If no positive change is detected in the user's sleep schedules or waking mood and alertness, device 100 may request input from the user to understand the problem better and make recommendations to help take some activities off their plate, to be able to make more time to rest and rejuvenate. Using motivational interviewing techniques, the processor 112 may determine the current state of the user and push them to make a change to their schedule or environment to positively impact their life, based on what has worked for them in the past.

By automatically and/or manually monitoring aspects of the user's life such as their home environment, car environment, wearable outputs, and emotional state, the processor 112 executing recommendation generation module 170 may be configured to make behaviour and recovery connections over time. Showing the user these correlations can help the user understand how their behaviour impacts mood, recovery, productivity at work and outside of work, as well as how their actions impact others around them. This helps in guiding the users to reach their goal of managing untimely work schedules and feeling involved in family events, while taking care of their overall health and wellness.

A testing trial was conducted to determine the efficacy of a sleep management system such as system 100 for personalized sleep-wake management in shift workers. 28 shift workers trialed the system for two weeks, following which they self-reported total sleep time, ability to fall asleep, sleep quality and overall recovery at baseline and post-application use. Measures of sleep-related impairments and mood (which included anxiety, stress and depression) were also measured at baseline and post-application use. Critical to quality performance indicators were used to determine effectiveness and engagement. Following 2 weeks of using system 100, the total sleep time reported by the trial participants was significantly increased (p=0.042), while participants also noted a significant improvement in their ability to fall asleep (p<0.001) and quality of sleep (p=0.001). Sleep-related impairments and measures of mood also significantly improved between baseline and post-application use (p<0.05), except depression, despite a trend toward improvement (p=0.071). Critical to quality measures all met the success criteria of >50%. The trial demonstrated the effectiveness of system 100 to improve sleep and health outcomes in shift workers.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method for improving data accuracy of sleep pattern data, the method comprising:
receiving first data relating to at least one sleep pattern metric;
receiving second data relating to the at least one sleep pattern metric, wherein the second data is data entered by a user;
determining the difference between the first data and the second data to calculate a data infidelity value; and
in response to the data infidelity value exceeding a predetermined threshold, prompting a user to enter third data relating to at least one metric.

2. The method of claim 1, wherein the first data is data entered by a user.

3. The method of claim 1, wherein the first data is sensor data received from at least one sensor.

4. The method of any one of claims 1 to 3, further comprising determining the difference between the first data, the second data and the third data to calculate an updated data infidelity value; and in response to the updated data infidelity value exceeding a predetermined threshold, repeating the steps of prompting the user to enter further data and calculating the updated data infidelity value until the updated data infidelity value does not exceed the predetermined threshold.

5. The method of any one of claims 1 to 4, further comprising prompting a user to enter second data relating to at least one metric, wherein the second data is received in response to the prompt.

6. The method of claim 5, wherein the prompting comprises presenting the user with a question, and the second data is the user's response to the question.

7. The method of any one of claims 1 to 6, wherein the second data is data received from a remote device comprising at least one sensor.

8. The method of any one of claims 1 to 7, wherein prompting the user to enter third data comprises presenting a modified question to the user, the modified question being based on a question previously presented to the user and having the same semantic meaning as the question previously presented to the user.

9. The method of claim 8, further comprising generating the modified question based on the question previously presented to the user using natural language processing techniques.

10. The method of claim 8 or claim 9, further comprising retrieving the modified question from a database of questions.

11. The method of any one of claims 1 to 10, further comprising processing the first data and the second data to map the data to the at least one sleep pattern metric.

12. The method of any one of claims 1 to 11, wherein the at least one sleep pattern metric comprises at least one of a time in bed metric, a total sleep time metric, a wake after sleep onset (WASO) metric, a sleep onset latency (SOL) metric, and a sleep efficiency metric.

13. The method of any one of claims 1 to 12, further comprising using at least one of the first data, second data and third data to determine a value for the at least one sleep pattern metric.

14. The method of claim 13, further comprising generating a sleep pattern recommendation for presenting to the user based on the determined value of the sleep pattern metric.

15. The method of any one of claims 1 to 14, further comprising prompting the user to confirm the accuracy of at least one of the first data, second data and third data.

16. The method of any one of claims 1 to 14, further comprising tracking any questions presented to the user that result in the user providing data having a high data infidelity value, to determine questions that lack clarity.

17. The method of claim 16, further comprising rewording any questions that result in the user providing data having a high data infidelity value.

18. The method of claim 16 or claim 17, further comprising tracking word combinations within questions presented to the user that result in the user providing data having a high data infidelity value, to determine word combinations that lack clarity.

19. A method for presenting sleep pattern recommendations to a user, the method comprising:
receiving sleep pattern data from a population;
performing clustering of the received sleep pattern data;
receiving sleep pattern data from a user;
identifying a cluster that is most closely associated with the sleep pattern data received from the user;
receiving a plurality of sleep pattern recommendations to provide to the user;
retrieving a sleep pattern recommendation order based on the identified cluster; and
ordering the plurality of sleep pattern recommendations based on the retrieved sleep pattern recommendation order.

20. The method of claim 19, further comprising presenting at least one of the plurality of sleep pattern recommendations to the user according to the retrieved sleep pattern recommendation order.

21. The method of claim 20, wherein the plurality of sleep pattern recommendations are presented to the user simultaneously.

22. The method of claim 20, wherein the plurality of sleep pattern recommendations are presented to the user sequentially.

23. The method of any one of claims 20 to 22, further comprising presenting the at least one of the plurality of sleep pattern recommendations to the user alongside a degree of effectiveness of the recommendation.

24. The method of any one of claims 19 to 23, further comprising pre-processing the sleep pattern data received from the user into a normalised data vector.

25. The method of any one of claims 19 to 24, wherein the clustering is performed using an agglomerative clustering technique.

26. The method of any one of claims 19 to 25, wherein the clustering is performed using at least one of partitioning clustering, k-means clustering and hierarchical clustering.

27. The method of any one of claims 19 to 26, further comprising masking the recommendations based on user data to avoid presenting the user with irrelevant or infeasible recommendations.

28. The method of claim 27, further comprising providing the user with an alternative recommendation to replace at least one masked recommendation.

29. The method of any one of claims 19 to 28, further comprising prompting the user to enter data relating to an effectiveness of the at least one recommendation.

30. The method of claim 29, wherein prompting the user to enter data relating to an effectiveness of the at least one recommendation comprise prompting the user to enter data relating to at least one of the user's waking mood, alertness and sleepiness after having adopted the at least one recommendation.

31. The method of claim 29 or claim 30, further comprising using the entered data to modify the sleep pattern recommendation order associated with the identified cluster.

32. The method of any one of claims 19 to 31, wherein the sleep pattern recommendations are generated according to the method of claim 14.

33. A method for improving sleep patterns in users, the method comprising:
receiving data relating to at least one sleep pattern metric from a first remote device;
processing the data to generate at least one sleep pattern recommendation;
processing the data to generate at least one instruction to a second remote device, to cause the second remote device to implement the recommendation;
displaying the at least one recommendation to the user; and
sending the at least one instruction to the second remote device.

34. The method of claim 33, further comprising pre-processing the data received from the first remote device to format the data to a common data format.

35. The method of claim 33 or claim 34, further comprising deriving at least one sleep pattern parameter from the data.

36. The method of any one of claims 33 to 35, wherein processing the data to generate at least one sleep pattern recommendation comprises using a decision tree.

37. The method of any one of claims 33 to 36, wherein processing the data to generate at least one sleep pattern recommendation comprises using a model driven recommendation model.

38. The method of claim 37, wherein the model driven recommendation model uses at least one of a bio-mathematical model and a biophysical model.

39. The method of claim 37 or claim 38, wherein the model uses a system of ordinary differential equations.

40. The method of claim 39, wherein the differential equations are based on neurobiological mechanisms of sleep and circadian regulation.

41. The method of any one of claims 33 to 40, wherein the first remote device comprises at least one of a home monitoring hub, a car monitoring hub, a recovery system, a wearable device, a smart cup, an augmented reality device, a virtual reality device, a biological data device, a bed partner input device, an emotion detection system, a manual entry system, a light sensor and a work place monitoring hub.

42. The method of any one of claims 33 to 41, wherein the second remote device comprises at least one of a change coaching system, a calendar input system, an augmented reality device, a virtual reality device, an engagement system, a biological feedback system, a home automation system, a communication system, a behaviour recommendation system, a long term connection system, and a car.

43. The method of any one of claims 33 to 42, wherein processing the data to generate at least one sleep pattern recommendation is performed according to the method of claim 14.

44. The method of any one of claims 33 to 43, wherein displaying the at least one recommendation to the user is performed according to the method of any one of claims 22 to 25.

45. A machine-readable medium storing non-transitory instructions which, when executed by one or more processors, cause an electronic apparatus to perform the method of any one of claims 1 to 44.

46. An apparatus, comprising processing circuitry and a machine-readable medium storing non-transitory instructions which, when executed by the processing circuitry, cause the apparatus to perform the method of any one of claims 1 to 44.
